(19) United States
(12) Patent Application Publication
(10) Pub. No.: US 2026/0050324 A1
(43) Pub. Date: Feb. 19, 2026

(54) APPARATUS AND METHOD FOR GENERATING A CONTROL SIGNAL FOR A MEDICAL INSTRUMENT OR A MEDICAL IMAGING DEVICE

(71) Applicant: KARL STORZ SE & Co. KG, Tuttlingen (DE)

(72) Inventor: Klaus Brüderle, Radolfzell (DE)

(73) Assignee: KARL STORZ SE & Co. KG, Tuttlingen (DE)

(21) Appl. No.: 19/303,867

(22) Filed: Aug. 19, 2025

(30) Foreign Application Priority Data
Aug. 19, 2024 (DE) 10 2024 123 657.4

(51) Int. Cl. G06F 3/01; A61B 17/00; H04N 13/344; H04N 13/383

(52) U.S. Cl. CPC G06F 3/013; A61B 17/00; H04N 13/344; H04N 13/383; A61B 2017/00216

(57) ABSTRACT

Apparatus, devices, and a method for generating a control signal for a medical instrument and/or a medical imaging device. The apparatus comprises: an accommodation detection device configured to detect an accommodation state of a human eye; and an output device configured to generate and output a control signal for the medical instrument and/or the medical imaging device on the basis of the detected accommodation state.

### CROSS-REFERENCE TO RELATED APPLICATIONS

The present application claims the benefit of German Patent Application 10 2024 123 657.4 filed on Aug. 19, 2024, having a title of, “Eyetrack-Cursorsteuerung für 3D Bildvisualisierungen (handfrei und gestenneutral)” (“Three-dimensional or spatial eye-tracking cursor control for 3D image visualizations (hands-free and gesture-neutral)”) and is incorporated herein by reference in its entirety.

### FIELD OF THE DISCLOSURE

The present invention relates to generating a control signal for a medical instrument and/or a medical imaging device.

### BACKGROUND

Imaging methods are frequently used in modern medicine. A medical imaging device is used to capture images of a medical scene. These images can provide a user (e.g., a surgeon), with information that the user could not visually perceive without aids, either because the images originate from a location that is not directly visible (e.g., from inside a patient) or because the information is based upon electromagnetic radiation at non-visible wavelengths.

The imaging device can often be controlled to display different regions, carry out magnifications, and the like. This requires a user interface by means of which a user can transmit control signals to the imaging device.

For imaging devices that display 2-dimensional (2-D) and 3-dimensional (3-D) image content, it is advantageous when the user interface is able to output control signals not only with regard to two dimensions (e.g., left/right and up/down as with a typical screen), but also with regard to a depth dimension (forwards/backwards).

Computer aided design (CAD) systems are known for navigating 3-D space by using special mice with 3-D control inputs. These 3-D mice require considerable practice for correct handling, as well as laboriously prepared for use in a medical or clinical environment—for example, placed in sterile packaging. In addition, control that is as intuitive and convenient as possible is preferred as it readily allows for the precise control of medical instruments and medical imaging devices.

### SUMMARY

It is with respect to the above issues and other problems that the embodiments presented herein were contemplated. As a general introduction, and in one embodiment, an improved apparatus and an improved method for generating a control signal for a medical instrument and/or a medical imaging device are provided.

According to a first aspect, an apparatus for generating a control signal for a medical instrument and/or a medical imaging device, comprising: an accommodation detection device configured to detect an accommodation state of a human eye; and an output device configured to generate and output a control signal for the medical instrument and/or the medical imaging device on the basis of the detected accommodation state.

“Accommodation” is generally understood to mean a dynamic adjustment of the refractive power of the human eye. Accommodation allows objects at different distances to be brought into focus of human vision. An accommodation state therefore refers to the a state of the human eye that is changed by an accommodation or is a result of accommodation. The accommodation state may include, for example, a curvature state of a lens of the human eye. In certain other embodiments, the accommodation state may be or include an absolute or relative thickness of the lens, a refractive power of the lens, and/or the like.

By using the accommodation state as a basis for control signals, these control signals can thus be provided in particular without the aid of the hands or other limbs. As a benefit of the embodiments herein, a user of a medical instrument and/or medical imaging device has both hands free while generating the control signals.

Furthermore, the present solution allows objectively improved human-machine interaction, since it is natural for any person—purely physiologically—to associate the depth dimension with the accommodation of the eye—for example, when trying to focus on distant objects with the naked eye. This natural and intuitive reaction is exploited as an input to the embodiments herein.

According to some preferred embodiments, variants, or refinements of embodiments, the output device is configured to generate the control signal such that it indicates positions, alignments, and/or movements in three orthogonal spatial directions—for example, to the left/right, upwards/downwards, and forwards/backwards. The control signal may generate a position (e.g., to mark a specific point in a real or virtual 3-dimensional space in a 3-D image), or whether the control signal should indicate an alignment (e.g., to pan a viewing angle of a medical imaging device such as a camera), and/or whether the control signal should indicate a movement (e.g., to control a medical probe, drone, camera, etc.).

In particular, the position, alignment, and/or movement with respect to a first of these three orthogonal spatial directions, particularly preferably a depth dimension, is preferably based upon the detected accommodation state. As already mentioned, this is a particularly intuitive link that objectively makes control easier for the user.

In other words, the accommodation state is to correlate with the position, alignment, and/or movement in the first spatial direction. The correlation may be proportional, polynomial (with polynomials of second or higher degree), exponential, or combinations thereof, such as according to a user-defined function. As a result, certain embodiments herein utilize high precision for position determinations or movements over short distances and rapid change/movement or position determinations or movements over longer distances.

The depth dimension refers to a dimension along which objects move in a line toward or away from the viewer without consideration of motion to the left or to the right, upwards or downwards. The “viewer,” as used herein may be either the user of the apparatus, who perceives a real or virtual 3-dimensional space, or a lens of an imaging device, which captures image data of a real 3-dimensional space. In another embodiment, fourth level and greater dimensions are provided as additional image sources, such as a second image (e.g., false color, x-ray, etc.) presented to the user.

In another embodiment, the apparatus only outputs control signals with respect to one or two orthogonal spatial directions, one of which-again, preferably the depth dimension-correlates with the detected accommodation state of the human eye.

According to some embodiments, variants, or refinements of embodiments, the apparatus can further comprise an orientation detection device which is configured to detect an orientation of a pupil of a human eye, wherein positions and/or movements in a second and a third of the three orthogonal spatial directions are based upon the detected orientation. For example, the orientation of the pupil is used to determine or control a position or movement in the left/right and upward/downward directions, while the detected accommodation state is used to determine or control a position, alignment, or movement in the forward/backward direction (i.e., in the depth dimension). In other words, the orientation of the pupil is detected and cursor control is implemented based on the detected pupil orientation.

As a benefit, embodiments herein provide a particularly intuitive control of medical instruments or medical imaging devices, in which a person only needs to indicate their control intention in the same way as they would do in order to take a closer look at an object of interest, namely, by moving the pupil to the left/right/upwards/downwards and by accommodation of their eye (i.e., by changing the accommodation state). In other words, a forward/backward indication is generated in the control signal on the basis of the focus (additionally or alternatively the width of focus) of the eye, and a left/right/upward/downward indication is generated on the basis of the orientation (additionally or alternatively, the alignment) of the eye.

The orientation detection device, a camera (such as the user eye-facing camera(s) of data glasses) is used in conjunction with methods for pupil tracking (eye tracking) as may be known in the prior art.

According to certain embodiments, variants, or refinements of embodiments, the accommodation detection device has a radiation source, a radiation detection unit, and a computing unit.

The radiation source is configured to emit measuring radiation into the human eye. The radiation detection unit is configured to detect response radiation that is reflected by the human eye and is based upon the emitted measuring radiation. The computing unit is designed to determine the accommodation state of the human eye on the basis of the detected response radiation.

Radiation generated by the radiation source enters the eye and is reflected as the response radiation in different ways, depending upon the current accommodation state of the eye, such as the curvature of the lens. Thus, the radiation detection unit can detect at least one property of the response radiation and, based thereon, determine the accommodation state.

According to certain embodiments, variants, or refinements of embodiments, the radiation source is designed to emit the measuring radiation at a wavelength of 600 nanometers to 800 nanometers, in particular of 625 nanometers to 700 nanometers, particularly preferably of 650 nanometers to 675 nanometers. These wavelengths have proven to be sufficient for good and precise detection, while at the same time being harmless to health and trouble-free for users.

According to certain embodiments, variants, or refinements of embodiments, the radiation source for emitting the measuring radiation has a punctiform, linear, or cruciform radiation emitter.

In another embodiment, narrowly emitted measuring radiation (e.g., emitted as a point) is used which has the advantage of running in a narrow channel and thus its reflection (i.e., the response radiation returned) and, as a result, is more easily detected. In addition, a radiation source with lower power is used while maintaining the same sensitivity of the radiation detection unit.

According to certain embodiments, variants, or refinements of embodiments, the apparatus comprises data glasses (which may also be known as “smart” glasses). At least the radiation source and/or the radiation detection unit is preferably formed on the data glasses, particularly preferably both. In this way, a user only needs to put on the data glasses and can thus generate control signals (e.g., control the medical instrument and/or the medical imaging device), without having to use their hands or feet, which would otherwise be required. The hands thus remain free, for example, to conduct a surgery or other medical procedure and handle one or more instruments—for example, a scalpel, an endoscope with a functional unit such as a jaw part, and the like.

According to certain embodiments, variants, or refinements of embodiments, the radiation source is configured to radiate the measuring radiation eccentrically into the human eye. This improves the detection of the accommodation state, as the accommodation state is detected better eccentrically (i.e., away from the center of the human eye (i.e., eyeball)). For example, measuring the radiation incident centrally into the eye would hit the lens substantially perpendicularly and would not be affected by different curvatures of the lens. The curvature of the lens (as a possible parameter of the accommodation state) has a significantly stronger effect on eccentrically incident measuring radiation.

According to certain embodiments, variants, or refinements of embodiments, the radiation source and/or the radiation detection unit is, or is configured for, stationary mounting in a room, such as an operating room, or other room in which medical instrument and/or medical imaging device is controlled.

According to certain embodiments, variants, or refinements of embodiments, the control signal is designed to set an image selection, a focus selection, and/or a subject selection in a 3-D representation. The 3-D representation can be a 3-D projection. A 3-D projection refers to the projection of a 3-D representation into or for the human eye—for example, through data glasses. In one embodiment, comprising data glasses, further comprise a projection unit for projecting the 3-D projection into the human eye. The 3-D projection can also be carried out by a head-up display, which can also be part of the apparatus.

According to certain embodiments, variants, or refinements of embodiments, the control signal is designed to carry out a position selection or object selection in a virtual reality environment or an augmented reality environment. The virtual reality environment or augmented reality environment can in turn be provided to a user by data glasses comprising the apparatus disclosed herein. It should be appreciated that, in other embodiment, the data glasses may be a visor, mask, or other wearable device operable to emit radiation to (or into) a user's eye (or eyes) and measure radiation reflected and/or refracted therefrom.

In another embodiment, a method is provided for generating a control signal for a medical instrument and/or a medical imaging device, comprising: detecting an accommodation state of a human eye; and generating and outputting a control signal for a medical instrument or medical imaging device on the basis of the detected accommodation state.

According to certain embodiments, variants, or refinements of embodiments, detecting the accommodation state comprises at least the steps of: generating measuring radiation; emitting the measuring radiation into the human eye; detecting reflected response radiation based upon the emitted measuring radiation; and determining the accommodation state on the basis of the detected response radiation.

In another embodiment, a computer program product is disclosed that comprises executable program code that is configured, when executed by a computing unit, to carry out the methods herein.

In another embodiment, a non-volatile, computer-readable data storage medium is disclosed that comprises executable program code that is configured, when executed by a computing unit, to carry out or control the methods disclosed herein.

The non-volatile, computer-readable data storage medium can comprise or consist of any type of computer memory, in particular semiconductor memory, such as, for example, a solid-state memory. The data carrier can also comprise or consist of a CD, a DVD, a Blu-ray disc, a USB memory stick, or the like.

In another embodiment, a data stream that comprises executable program code or is configured to generate executable program code that is configured, when executed by a computing unit, to carry out or control the methods disclosed herein.

Further advantageous variants, options, embodiments, and modifications are disclosed in the accompanying figures, the detailed description, and the claims. However, it is self-evident that the detailed description and specific examples, while indicating preferred embodiments, are presented for illustration only, since various changes and modifications may be apparent to a person skilled in the art and applied without departing from the scope of the embodiments herein.

The phrases “at least one,” “one or more,” and “and/or” are open-ended expressions that are both conjunctive and disjunctive in operation. For example, each of the expressions “at least one of A, B, and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

The term “computer-readable medium,” as used herein, refers to any tangible storage that participates in providing instructions to a microprocessor for execution. Such a medium may take many forms, including, but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, a RAM, a PROM, an EPROM, a FLASH-EPROM, a solid-state medium like a memory card, any other memory chip or cartridge, or any other medium from which a computer can read. When the computer-readable media is configured as a database, it is to be understood that the database may be any type of database, such as relational, hierarchical, object-oriented, and/or the like. Accordingly, the disclosure is considered to include a tangible storage medium and prior art-recognized equivalents and successor media, in which the software implementations of the present disclosure are stored.

While machine-executable instructions may be stored and executed locally to a particular machine (e.g., personal computer, mobile computing device, laptop, etc.), it should be appreciated that the storage of data and/or instructions and/or the execution of at least a portion of the instructions may be provided via connectivity to a remote data storage and/or processing device or collection of devices, commonly known to as “the cloud,” but may include a public, private, dedicated, shared and/or other service bureau, computing service, and/or “server farm.”

The terms “determine,” “calculate,” and “compute,” and variations thereof, as used herein, are used interchangeably and include any type of methodology, process, mathematical operation, or technique.

The term “module,” as used herein, refers to any known or later-developed hardware, software, firmware, artificial intelligence, fuzzy logic, or combination of hardware and software that is capable of performing the functionality associated with that element. Also, while the disclosure is described in terms of exemplary embodiments, it should be appreciated that other aspects of the disclosure can be separately claimed.

### BRIEF DESCRIPTION OF THE DRAWINGS

Individual embodiments of the present disclosure will be explained in detail with reference to the following figures. The components in the drawings are not necessarily to scale, but serve to illustrate the principles of the present disclosure. Parts in the different figures that correspond to the same elements or method steps have been given the same reference signs in the figures. The numbering of method steps initially serves only to distinguish them and does not necessarily imply a corresponding order, although one variant is to carry out the steps in the order in which they are numbered. Multiple steps can also be carried out in an overlapping or simultaneous manner.

The present disclosure is described in conjunction with the appended figures:

FIG. 1 depicts a schematic representation of an apparatus in accordance with embodiments of the present disclosure;

FIG. 2 depicts details of the apparatus from FIG. 1 in accordance with embodiments of the present disclosure;

FIG. 3 is a schematic flow diagram for explaining a method in accordance with embodiments of the present disclosure;

FIG. 4 is a schematic block diagram for explaining a computer program product in accordance with embodiments of the present disclosure; and

FIG. 5 is a schematic block diagram for explaining a data storage medium in accordance with embodiments of the present disclosure.

### DETAILED DESCRIPTION

The ensuing description provides embodiments only and is not intended to limit the scope, applicability, or configuration of the claims. Rather, the ensuing description will provide those skilled in the art with an enabling description for implementing the embodiments. It will be understood that various changes may be made in the function and arrangement of elements without departing from the spirit and scope of the appended claims.

Any reference in the description comprising an element number, without a subelement identifier when a subelement identifier exists in the figures, when used in the plural, is intended to reference any two or more elements with a like element number. When such a reference is made in the singular form, it is intended to reference one of the elements with the like element number without limitation to a specific one of the elements. Any explicit usage herein to the contrary or providing further qualification or identification shall take precedence.

The exemplary systems and methods of this disclosure will also be described in relation to analysis software, modules, and associated analysis hardware. However, to avoid unnecessarily obscuring the present disclosure, the following description omits well-known structures, components, and devices, which may be omitted from or shown in a simplified form in the figures or otherwise summarized.

For purposes of explanation, numerous details are set forth in order to provide a thorough understanding of the present disclosure. It should be appreciated, however, that the present disclosure may be practiced in a variety of ways beyond the specific details set forth herein.

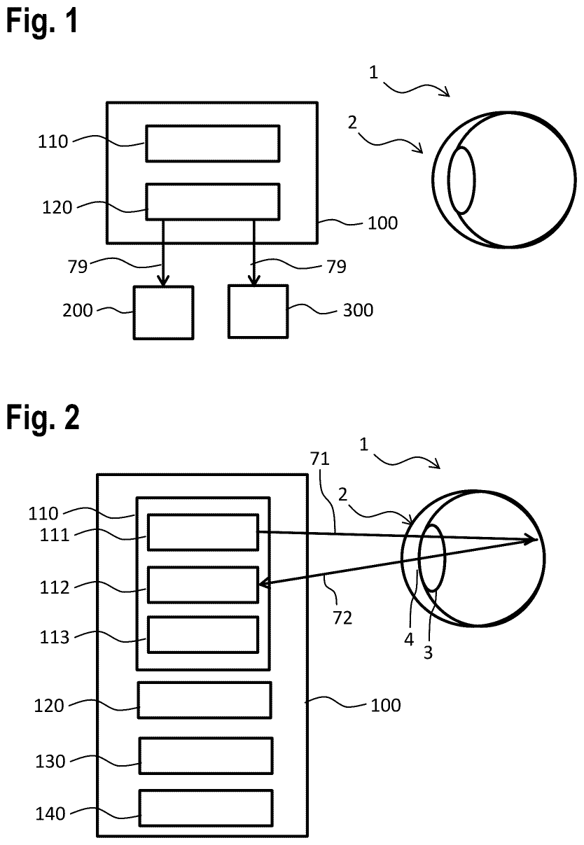

FIG. 1 shows a schematic representation of an apparatus according to an embodiment of the present disclosure. In one embodiment, an apparatus 100 is provided to generate a control signal 79 for a medical instrument 200 and/or a medical imaging device 300. The medical instrument 200 can, for example, be a surgical robot or a controllable camera system. The control signal 79 can, for example, control an alignment, a position, and/or a direction of movement in the medical instrument 200 or the medical imaging device 300. The apparatus 100 according to certain embodiments comprises the medical instrument 200 to be controlled and/or the medical imaging device 300 to be controlled.

In another embodiment, the medical imaging device 300 is a surgical camera system that displays image content, which may be combined. Image contents may be displayed side by side, on top of one another, or inside one another. Imaged subjects may be captured three-dimensionally (for example, by surgical camera systems that comprise stereoscopic cameras). Additional multi-dimensionality may be provided through additional channels. For example, a first channel may comprise a true color image, a second channel a false color image, and/or the like. A dimension in which or for which the control signal 79 can effect control can thus also be the dimension of the channel numbers.

The surgical camera system can have a camera, a camera controller for processing the images captured by the camera, and a display device for displaying the captured and/or processed images. The camera may be fastened to a microscope or a movable frame and/or be an endoscopic camera. The medical imaging device 300 can thus be, in particular, an endoscopic imaging device.

The control signal 79 can carry out one or more of the following controls: an image selection in a 3-D representation, a focus selection in a 3-D representation, and/or a subject selection in a 3-D representation, wherein the 3-D representation can in each case be a 3-D projection—for example, into the eye. Alternatively or additionally, the control signal may carry out a position selection or object selection in a virtual reality environment or an augmented reality environment.

The apparatus 100 comprises an accommodation detection device 110, which is configured to detect an accommodation state 2 of a human eye 1, and an output device 120, which is configured to generate and output the control signal 79 for the medical instrument 200 and/or the medical imaging device 300 on the basis of the detected accommodation state 2.

In one embodiment, the output device 120 generates the control signal 79 such as to indicates positions, alignments, and/or movements in one to three orthogonal spatial directions. The position, alignment, and/or movement with respect to a first of these orthogonal spatial directions, in particular a depth dimension, is advantageously based upon the detected accommodation state 2.

For example, the accommodation state 2 can comprise or consist of a curvature state of a lens 3 of the eye 1. A first accommodation state 2, i.e., in this example, a first curvature state of the lens 3, can be defined as the zero position, so that accommodation states 2 with a greater curvature of the lens 3 than the first curvature state can be associated with coordinates of the depth dimension along a first direction (e.g., negative z-direction), and accommodation states 2 with a lesser curvature of the lens 3 than the first curvature state can be associated with coordinates of the depth dimension along a second direction opposite to the first direction (e.g., positive z-direction).

The negative z-direction is typically a direction away from the viewer (camera lens, data glasses, eye 1, etc.), while the positive z-direction points toward the viewer. Here, an orthogonal tripod is assumed in which a positive x-direction points from left to right, a positive y-direction points from bottom to top, and finally the positive z-direction points from back to front, i.e., toward the viewer of the tripod.

Depending upon the application, these associated coordinates can then be converted by the output device 120 into a corresponding control signal 79, which can indicate or control, for instance, a position, movement, or alignment according to the corresponding coordinate. Thus, by accommodation of a user's eyes 1 for longer distance vision, a user can generate a control signal 79 using the apparatus 100, which control signal, for example, controls a medical imaging device 300 to display more distant details in more detail—for example, by zooming in and/or moving its input optics. As an example, the coordinate of the depth dimension can indicate a projection plane for cursor control in a 3-D image, in particular control a medical imaging device 300 to select this projection plane, to display a sectional view through this plane, or the like.

FIG. 2 depicts a schematic representation of the apparatus 100 with details in accordance with the disclosure. In one embodiment, apparatus 100 comprises an orientation detection device 130, which is configured to detect an orientation of a pupil 4 of the human eye 1—for example, by means of “eye-tracking” methods known in the prior art. Advantageously, positions, alignments, and/or movements in a second and optionally a third of the three orthogonal spatial directions indicated (or displayed) by the generated control signal 79 may be based upon the detected orientation of the pupil 4. With reference to the orthogonal tripod x, y, z described above, positions, alignments, or movements in the x-direction and/or y-direction can thus be displayed or controlled by the control signal 79 on the basis of the orientation of the pupil 4.

Thus, for example, a user can look to the left to generate a control signal 79 that causes a movement of a cone of vision of a medical imaging device 300 to the left, and in the process bend the lens 3 (through conscious or unconscious accommodation) such that the control signal 79 is additionally generated such that the cone of vision is moved forwards, or the like.

Certain details of the accommodation detection device 110 are described below. These embodiments may be provided together with the orientation detection device 130, but also separately. In other words, the accommodation detection device 110 with the details described below can also be provided in an apparatus 100 that generates the control signal 79 such that it only displays or indicates positions, alignments, or movements in a single direction/dimension, in particular the depth dimension.

The accommodation detection device 110 can in particular have a radiation source 111, a radiation detection unit 112, and a computing unit 113. Here, the radiation source 111 is configured to generate measuring radiation 71 and to emit it into the human eye 1—for example, at a wavelength of 600 nanometers to 800 nanometers, in particular of 625 nanometers to 700 nanometers, particularly preferably of 650 nanometers to 675 nanometers. As already explained above, these wavelengths are particularly suitable for being reflected well by the human eye without causing annoyance or any damage.

The radiation source 111 can have a punctiform or linear emitter, which may be arranged on or in data glasses.

The radiation detection unit 112 is configured to detect response radiation 72 reflected by the human eye 1 and based upon the emitted measuring radiation 71. Such response radiation 72 is generated, for example, by measuring radiation 71 reflected by the retina. The wavelengths mentioned above are particularly suitable for this purpose. If both the radiation source 111 and the radiation detection unit 112 are arranged on data glasses, in particular integrated into the data glasses, they can advantageously be arranged relative to one another such that at least a part (preferably the majority) of the measuring radiation 71 reflected by the human eye 1 impinges on the radiation detection unit 112 as response radiation 72.

The radiation detection unit 112 may be embodied as a semiconductor photosensor or the like.

Advantageously, the radiation source 111 emits the measuring radiation 71 into the human eye 1 eccentrically. Alternatively or additionally, the radiation source 111 and the radiation detection unit 112 are arranged (in particular with respect to data glasses on which they are arranged) such that the response radiation 72 leaves the human eye eccentrically. As explained above, for example, the curvature of the lens 3 is more pronounced at its radial edges than in its radial center. Therefore, the measuring radiation 71 or the response radiation 72 is subject to greater changes (e.g., greater refraction), when it passes through the lens 3 eccentrically, i.e., closer to its radial edges than to its radial center, than when it passes through the lens 3 centrally. Accordingly, when the measuring radiation 71 enters eccentrically and/or when the response radiation 72 exits eccentrically, the response radiation 72 contains, ceteris paribus, more clearly defined information about the accommodation state 2, which can accordingly be determined with less effort, more precisely, and more accurately.

The computing unit 113 is provided for this purpose. This is designed to determine the accommodation state 2 of the human eye 1 on the basis of the detected response radiation 72. In the example used above, for example, a comparison of the direction of emission (or other properties) of the measuring radiation 71 (known to the computing unit 113) with the direction of incidence (or other properties) of the response radiation 72 by the computing unit 113 may be used to determine a refraction through the lens 3, which in turn allows a conclusion to be drawn about the curvature of the lens 3 and thus its accommodation state 2.

However, as explained above, other variants are also possible for determining different accommodation states of the eye 1. For example, the accommodation detection device 110 may be configured to capture an image of the human eye 1 and to evaluate the captured image in order to determine the accommodation state 2 on the basis of the image data. For this purpose, the computing unit 113 can, for example, implement a machine learning model (e.g., an artificial neural network) that is trained to determine the accommodation state 2 of the eye 1 from such images of the human eye 1.

Previously, an embodiment was described in which at least the radiation source 111 and the radiation detection unit 112 (and, optionally, additionally the computing unit 113) are attached to data glasses or integrated into data glasses of the apparatus 100. In some variants, even the entire apparatus 100 is integrated into data glasses. In other variants, the data glasses can have a wireless or wired transmitter of a communications device 140 of the apparatus 100, by means of which it communicates with other components of the apparatus 100—for example, the computing unit 113.

In another embodiment, the computing unit 113 is any device that is designed and configured for digital computing, in particular for running software, an application, or an algorithm. The computing unit 113 can, for example, comprise at least one processor unit (e.g., at least one CPU), at least one graphics processor unit (e.g., at least one GPU), at least one field-programmable gate array (FPGA), and/or at least one application-specific integrated circuit (ASIC), and/or any combination of the aforementioned elements. The computing unit 113 can also comprise a working memory and/or a non-volatile data memory, which are operatively linked to one another and/or to some or all of the aforementioned elements. The computing unit 113 may be implemented partially or entirely in a local unit (for example, a personal computer, PC, a laptop, a notebook, or the like) and/or partially or entirely in a distributed system.

In another embodiment, the computing unit 113 is provided, for example, by a server and/or a cloud computing platform. The computing unit 113 can also be integrated, together with the output device 120, into the medical instrument 200 to be controlled or the medical imaging device 300 to be controlled. In this way, data glasses can be kept particularly light and comfortable to wear, for example, by having only the radiation source 111, the radiation detection unit 112, and a part (in particular a transmitter) of the communications device 140 for communication with the remaining components of the apparatus 100. The output signals of the radiation detection unit 112 can then, for example, be processed only after they have been transmitted, by means of the communications device 140, to the separately arranged computing unit 113, which is connected there to the output device 120. For this purpose, the communications device 140 has at least one transmitter on the data glasses and a receiver on the computing unit 113—advantageously, a transceiver in each case. The computing unit 113 may also implement individual devices or units of the apparatus 100—for example, the output device 120.

However, embodiments are also possible in which no data glasses are present, or the data glasses have fewer elements/devices/units, or others. In some embodiments, for example, the radiation source 111 is fixedly mountable or mounted in a room (e.g., in an operating room or examination room), while the radiation detection unit 112 is mounted or arranged on the data glasses or another object worn by a user (“wearable”)—for example, a headband. In this case, an emitter of the radiation source 111 can have a geometric pattern other than a point—for example, a line shape, a grid shape, or a cross shape.

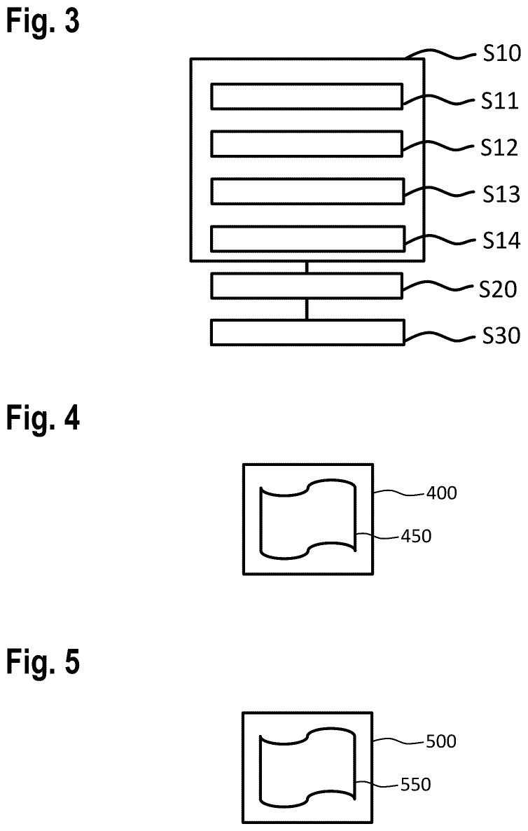

FIG. 3 shows a schematic flow diagram for explaining a method in accordance with the disclosure. In one embodiment, a method is disclosed for generating a control signal for a medical instrument 200 and/or a medical imaging device 300, in particular for 3-D control. The method may be carried out with the apparatus 100 a and is adaptable according to variants, options, embodiments, and refinements of embodiments described with reference to the apparatus 100, and vice versa. However, the method can also be carried out independently of the apparatus 100.

In a first step S10 of the method, an accommodation state 2 of a human eye 1 is detected—for example, as described above with reference to the accommodation detection device 110.

Accordingly, the detection S10 of the accommodation state 2 can, for example, comprise the following sub-steps S11, S12, S13, and S14.

In an optional sub-step S11, measuring radiation 71 is generated, and in an optional sub-step S12, the generated measuring radiation 71 is emitted into the human eye 1—for example, as described above with reference to the radiation source 111.

Accordingly, the measuring radiation 71 can preferably be generated S11 at a wavelength of 600 nanometers to 800 nanometers, in particular of 625 nanometers to 700 nanometers, particularly preferably of 650 nanometers to 675 nanometers.

In an optional sub-step S13, reflected response radiation 72 based upon the emitted measuring radiation 71 (e.g., generated by it or at least partially consisting of it) is detected—for example, as described above with reference to the radiation detection unit 112. In an optional sub-step S14, the accommodation state 2 is determined on the basis of the detected response radiation 72—for example, as described above with reference to the computing unit 113.

The emission S12 preferably takes place eccentrically with respect to the human eye 1, in particular such that the measuring radiation 71 and/or the response radiation 72 passes through a lens 3 of the human eye closer to radial edges of the lens 3 than to the radial center of the lens 3.

As previously explained above, the detection S10 of the accommodation state 2 may also be carried out in other ways, e.g., via image capture and image analysis, in particular using a machine learning model.

In any case, in a step S20, a control signal 79 for a medical instrument 200 or a medical imaging device 300 is generated and output on the basis of the detected accommodation state 2.

In a step S30, the medical instrument 200 or the medical imaging device 300 may be controlled by means of the control signal 79—for example, for one of the many applications described above.

FIG. 4 shows a schematic block diagram of a computer program product 400 in accordance with the disclosure. The computer program product 400 comprises executable program code 450 that is configured, when executed (e.g., by a computing unit 113), to carry out or control the method according to an embodiment of the present disclosure—for example, according to FIG. 3.

FIG. 5 shows a schematic block diagram of a non-volatile, computer-readable data storage medium in accordance with the disclosure. The data storage medium 500 comprises executable program code 550 that is configured, when executed (e.g., by a computing unit 113), to carry out or control the method according to an embodiment of the present disclosure—for example, according to FIG. 3.

The non-volatile, computer-readable data storage medium 500 can, for example, be designed as or have a semiconductor memory, e.g., an SSD memory chip. The data storage medium 500 can also have or comprise a CD, DVD, Blu-ray, or a magnetic storage device.

In the foregoing description, for the purposes of illustration, methods were described in a particular order. It should be appreciated that in alternate embodiments, the methods may be performed in a different order than that described without departing from the scope of the embodiments. It should also be appreciated that the methods described above may be performed as algorithms executed by hardware components (e.g., circuitry) purpose-built to carry out one or more algorithms or portions thereof described herein. In another embodiment, the hardware component may comprise a general-purpose microprocessor (e.g., CPU, GPU) that is first converted to a special-purpose microprocessor. The special-purpose microprocessor then having had loaded therein encoded signals causing the, now special-purpose, microprocessor to maintain machine-readable instructions to enable the microprocessor to read and execute the machine-readable set of instructions derived from the algorithms and/or other instructions described herein. The machine-readable instructions utilized to execute the algorithm(s), or portions thereof, are not unlimited but utilize a finite set of instructions known to the microprocessor. The machine-readable instructions may be encoded in the microprocessor as signals or values in signal-producing components and included, in one or more embodiments, voltages in memory circuits, configuration of switching circuits, and/or by selective use of particular logic gate circuits. Additionally or alternative, the machine-readable instructions may be accessible to the microprocessor and encoded in a media or device as magnetic fields, voltage values, charge values, reflective/non-reflective portions, and/or physical indicia.

In another embodiment, the microprocessor further comprises one or more of a single microprocessor, a multi-core processor, a plurality of microprocessors, a distributed processing system (e.g., array(s), blade(s), server farm(s), “cloud”, multi-purpose processor array(s), cluster(s), etc.) and/or may be co-located with a microprocessor performing other processing operations. Any one or more microprocessor may be integrated into a single processing appliance (e.g., computer, server, blade, etc.) or located entirely or in part in a discrete component connected via a communications link (e.g., bus, network, backplane, etc. or a plurality thereof).

Examples of general-purpose microprocessors may comprise, a central processing unit (CPU) with data values encoded in an instruction register (or other circuitry maintaining instructions) or data values comprising memory locations, which in turn comprise values utilized as instructions. The memory locations may further comprise a memory location that is external to the CPU. Such CPU-external components may be embodied as one or more of a field-programmable gate array (FPGA), read-only memory (ROM), programmable read-only memory (PROM), erasable programmable read-only memory (EPROM), random access memory (RAM), bus-accessible storage, network-accessible storage, etc.

These machine-executable instructions may be stored on one or more machine-readable mediums, such as CD-ROMs or other type of optical disks, floppy diskettes, ROMs, RAMS, EPROMS, EEPROMs, magnetic or optical cards, flash memory, or other types of machine-readable mediums suitable for storing electronic instructions. Alternatively, the methods may be performed by a combination of hardware and software.

Also, it is noted that the embodiments were described as a process, which is depicted as a flowchart, a flow diagram, a data flow diagram, a structure diagram, or a block diagram. Although a flowchart may describe the operations as a sequential process, many of the operations may be performed in parallel or concurrently. In addition, the order of the operations may be re-arranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

Furthermore, embodiments may be implemented by hardware, software, firmware, middleware, microcode, hardware description languages, or any combination thereof. When implemented in software, firmware, middleware or microcode, the program code or code segments to perform the necessary tasks may be stored in a machine-readable medium, such as a storage medium. A microprocessor(s) may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

Embodiments herein comprising software are executed, or stored for subsequent execution, by one or more microprocessors and are executed as executable code. The executable code being selected to execute instructions that comprise the particular embodiment. The instructions executed being a constrained set of instructions selected from the discrete set of native instructions understood by the microprocessor and, prior to execution, committed to microprocessor-accessible memory. In another embodiment, human-readable “source code” software, prior to execution by the one or more microprocessors, is first converted to system software to comprise a platform (e.g., computer, microprocessor, database, etc.) specific set of instructions selected from the platform's native instruction set.

While illustrative embodiments of the disclosure have been described in detail herein, it is to be understood that the inventive concepts may be otherwise variously embodied and employed, and that the appended claims are intended to be construed to include such variations, except as limited by the prior art.

What is claimed is:

1. An apparatus for generating a control signal, comprising:
an accommodation detection device that detects an accommodation state of a human eye; and
an output device that generates and outputs the control signal in accordance with the detected accommodation state; and
wherein the output device outputs the control signal to at least one of a medical instrument or a medical imaging device.

2. The apparatus of claim 1, wherein:
the output device generates the control signal to indicate at least one of a position, alignment, or movement in three orthogonal spatial directions, and wherein the at least one position, alignment, or movement with respect to a first of these three orthogonal spatial directions, in particular a depth dimension, is based upon the detected accommodation state.

3. The apparatus of claim 2, further comprising:
an orientation detection device that detects an orientation of a pupil of the human eye; and
wherein the at least one of position, alignment, or movement in a second and a third of the three orthogonal spatial directions are based upon the detected orientation.

4. The apparatus of claim 1, wherein the accommodation detection device has a radiation source, a radiation detection unit, and a computing unit, wherein the radiation source emits measuring radiation into the human eye, wherein the radiation detection unit detects response radiation reflected by the human eye and based upon the emitted measuring radiation, and wherein the computing unit determines the accommodation state of the human eye on the basis of the detected response radiation.

5. The apparatus of claim 4, wherein the radiation source emits the measuring radiation at a wavelength of 600 nanometers to 800 nanometers.

6. The apparatus of claim 5, wherein the radiation source emit the measuring radiation at a wavelength of 625 nanometers to 700 nanometers.

7. The apparatus of claim 6, wherein the radiation source emit the measuring radiation at a wavelength of 650 nanometers to 675 nanometers.

8. The apparatus of claim 4, wherein the radiation source has a punctiform, linear, or cruciform radiation emitter for emitting the measuring radiation.

9. The apparatus of claim 1, wherein the apparatus has data glasses, and at least one of the radiation source or the radiation detection unit is formed on the data glasses.

10. The apparatus of claim 9, wherein the radiation source radiates the measuring radiation into the human eye eccentrically.

11. The apparatus of claim 4, wherein the at least one of the radiation source or the radiation detection unit is configured for stationary mounting in a room.

12. The apparatus of claim 1, wherein the control signal sets at least one of an image selection, a focus selection, or a subject selection in a 3-D representation.

13. The apparatus of claim 1, wherein the control signal carries out a position selection or object selection in a virtual reality environment or an augmented reality environment.

14. A method for generating a control signal, comprising:
detecting an accommodation state of a human eye;
generating and outputting a control signal for a medical instrument or a medical imaging device on the basis of the detected accommodation state; and
providing the control signal to at least one of a medical instrument or a medical imaging device.

15. The method of claim 14, wherein the detection of the accommodation state comprises at least:
generating measuring radiation; emitting the measuring radiation into the human eye;
detecting reflected response radiation based upon the emitted measuring radiation; and
determining the accommodation state on the basis of the detected response radiation.

16. A wearable device, comprising:
a support to retain the wearable device on a user;
the support further retaining an accommodation detection device that detects an accommodation state of an eye of the user;
an output device that generates and outputs a control signal in accordance with the detected accommodation state; and
providing the output control signal to at least one of a medical instrument or a medical imaging device.

17. The wearable device of claim 16, wherein:
the output device generates the control signal to indicate at least one of a position, alignment, or movement in three orthogonal spatial directions, and wherein the at least one position, alignment, or movement with respect to a first of these three orthogonal spatial directions, in particular a depth dimension, is based upon the detected accommodation state.

18. The wearable device of claim 17, further comprising:
an orientation detection device that detects an orientation of a pupil of the eye; and
wherein the at least one of position, alignment, or movement in a second and a third of the three orthogonal spatial directions are based upon the detected orientation.

19. The wearable device of claim 16, wherein the accommodation detection device has a radiation source, a radiation detection unit, and a computing unit, wherein the radiation source emits measuring radiation into the eye, wherein the radiation detection unit detects response radiation reflected by the eye and based upon the emitted measuring radiation, and wherein the computing unit determines the accommodation state of the eye on the basis of the detected response radiation.

20. The wearable device of claim 19, wherein the radiation source emits the measuring radiation at a wavelength of 600 nanometers to 800 nanometers.
